(19) United States
(12) Patent Application Publication
(10) Pub. No.: US 2004/0131598 A1
(43) Pub. Date: Jul. 8, 2004

(54) Cancer therapy

(76) Inventors: Sabrina Tafuro, Oxford (GB); Ute-Christiane Meier, Oxford (GB); Andrew James McMichael, Oxford (GB); John Irving Bell, Oxford (GB); Guy Layton, Oxon (GB); Michael Hunter, Aylesbury (GB)

Correspondence Address: Hamilton Brook Smith & Reynolds, P O Box 9133, 530 Virginia Road, Concord, MA, 01742-9133, US

(21) Appl. No.: 10/415,841

(22) PCT Filed: Nov. 1, 2001
(86) PCT No.: PCT/GB01/04844

(30) Foreign Application Priority Data
Nov. 2, 2000 (GB) 0026812.8

(51) Int. Cl. A61K 48/00; C07H 21/04

(52) U.S. Cl. 424/93.21; 536/23.1

(57) ABSTRACT

The present invention relates to polynucleotides for use in cancer therapy. In particular, the invention provides a polynucleotide capable of expressing an epilope-β₂m fusion protein; for use in the generation of cytotoxic T lymphocyte (CTL) responses against a tumour, and a polynucleotide capable of expressing an epitope-β₂m fusion protein; for use in a method of restoring antigen presentation in the tumour of a host.

### FIELD OF THE INVENTION

The invention relates to a polynucleotide for use in cancer therapy.

### BACKGROUND TO THE INVENTION

Tumour cells are attacked by cytotoxic T lymphocytes (CTL) that recognise peptides presented by HLA class I molecules on the surface of the tumour cell. In many tumours down regulation of surface expression of class I MHC occurs allowing the tumours to escape from attack by CTL.

### SUMMARY OF THE INVENTION

The inventors have now shown that deliver of β₂m covalently linked to a peptide epitope restores surface class I molecule expression in a cell line. They have shown that expression in a tumour cell of the β₂m fusion protein from a polynucleotide allows correct assembly of the class I molecule, its expression on the surface, and that the tumour cell becomes susceptible to attack by CTL. The β₂m fusion protein may comprise a non-cancer epitope causing the tumour to become susceptible to attack by CTL which recognise non-cancer epitopes.

Accordingly the invention provides a polynucleotide capable of expressing an epitope-β₂m fusion protein; for use in the generation of cytotoxic T lymphocyte (CTL) responses against a tumour. Also provided is a polynucleotide capable of expressing an epitope-β₂m fusion protein; for use in a method of restoring antigen presentation in the tumour of a host.

### DETAILED DESCRIPTION OF THE INVENTION

The invention relates to the treatment of cancer by expressing in tumour cells a peptide epitope-β₂m fusion protein. Expression of the fusion protein in tumour cells may lead to an increased cytotoxic T lymphocyte (CTL) response against the cell, and may restore or improve antigen presentation in the cell.

Typically the tumour is a malignant tumour, for example, a solid tumour such as a gastrointestinal tumour, like colorectal cancer, gastro-esophageal cancer, cancer of liver and biliary tract, pancreatic cancer; prostatic cancer, testicular cancer, lung cancer, breast cancer, malignant melanoma, mesothelioma, brain tumours (such as glioma and astrocytomas), ovarian cancer, uterine cancer including cervical cancer, cancer of the head and neck (e.g. laryngeal), bladder cancer, Kaposi sarcoma, including AIDS-related Kaposi sarcoma, sarcomas and osteosarcoma, renal carcinoma; and hematopoietic malignant tumours such as leukemia and lymphoma, including AIDS-related lymphomas.

A CTL response may be generated against a tumour by expression in the tumour cells of a peptide epitope-Pm fusion protein. Preferably, the CTL response is sufficient to induce antigen specific killing of the tumour cells which express the fusion protein. The effectiveness of a CTL response may be measured in vitro. For example, a cytotoxic T cell assay may be carried out by CD8+ T cell-restricted IFN-γ ELISPOT and functional assays which assess antigen-specific CD8+ T cell killing by release of a measurable label, (e.g. Europium or Chromium) from killed target cells. An increase in the number (ELISPOT) or killing reflects an increase in cytotoxic T lymphocytes.

The present invention provides a means of treating a tumour by expressing in the tumour cells an epitope-β₂m fusion protein. Expression of the fusion protein in a tumour cell may restore surface class I antigen presentation. Antigen presentation may be reduced in a tumour cell due to impairment of one or more components of the class I antigen presentation pathway. Typically the impairment is due to a mutation which causes a reduced amount of the component to be expressed or causes an altered form of the component (typically differently glycosylated or mutated, e.g. truncated) to be expressed which has reduced or no activity. The impairment may be in the transport of the component. In one embodiment the component is expressed from a gene at reduced levels due to changes in methylation or chromatin structure, e.g. MHC class I genes.

The component is typically involved in (and therefore the impairment typically affects) processing (proteolysis) of proteins to form peptides, transport of the peptides to the endoplasmic reticulum or formation or transport of the class I molecule/peptide complex. Typically the component which is impaired is the class I heavy chain TAP or β₂m.

Restoration of antigen presentation may be seen as an increase or improvement in antigen presentation in the tumour cell. Antigen presentation may be restored to a level which is substantially the same as, or greater than, that expected in an equivalent non-tumour cell. Antigen presentation may be restored to a level which is lower than that expected in an equivalent non-tumour cell, but greater than that present in the cell before expression of the fusion protein.

Successful expression of a fusion protein in a tumour cell may lead to correct assembly of class I MHC within the cell, expression on the surface of the cell and presentation of the epitope at the surface of the cell, therefore rendering the cell susceptible to attack by cytotoxic T lymphocytes (CTL).

The fusion protein is capable of binding a class I heavy chain to form a class I molecule. Generally the class I molecule has a conformation substantially similar to the class I molecule formed by that heavy chain and the β₂m of the host. Thus a class I molecule containing the fusion protein will bind a conformation dependent antibody (e.g. W6/32) and/or can present epitope to the CTL of the host. Typically the fusion protein is capable of restoring surface class I molecule expression in T2 cells (e.g. detectable by use of W6/32 antibody).

The epitope of the fusion protein is capable of binding a class I molecule, such as a HLA-A, -B or -C molecule. Typical class I molecules which the epitope can bind include HLA-A1, -A2, -A3, -A24, -A31, -A68, -B7, -B8, -B44, -Cw6 and -Cw16. The epitope generally has a length of 8, 9, 10, 11 or 12 amino acids. The epitope may be a cancer epitope, i.e. an epitope only presented by tumour cells, typically an epitope which is recognised by tumour specific CTL in cancer patients. The epitope may be a neoantigen or from a protein which is expressed in increased amounts in a tumour cell. The epitope may be from any of the proteins which are discussed below that are expressed in a different form or in an increased amount in tumour cells.

In a preferred embodiment the epitope is a non-cancer epitope. Such an epitope is typically one which does not occur in a host protein, and is preferably an epitope which occurs in an intracellular pathogen of the host, such as a virus (e.g. EBV, CMV or influenza virus. Preferably the epitope is from any one of the proteins mentioned in Table 1, such as in, or represented by, any of the specific epitopes listed in Table 1.

The epitope is connected to the β₂m by a protein linker. Typically, the linker comprises amino acids that do not have bulky side groups and therefore do not obstruct the folding of the protein. The linker generally allows the epitope to bind to the groove of class I molecule formed after the fusion protein binds to the heavy chain. The linker typically has a length of 5 to 20 amino acids, such as 10 to 18 or 14 to 16, preferably 15 amino acids. Typically at least 50%, 70% or 90% of the amino acids in the linker are serine or glycine. The linker is generally connected to the N terminus of the 3₂m (in the form of a fusion protein). In a preferred embodiment the linker is or comprises GGSGGGGSGGSGGSG.

The term β₂m encompasses any beta-2 microglobulin protein, for example, a mammalian beta-2 microglobulin protein such as human or murine beta-2 microglobulin. The β₂m sequence is preferably the same or homologous to the β₂m of the host, or a fragment thereof. Thus, typically the β₂m has a length of at least 30, 40, 50 or 80 amino acids.

Table 5 shows the polynucleotide sequence of human β₂m and the amino acid sequence which it encodes. Preferably the β₂m is a full length β₂m protein such as the human beta-2 microglobulin shown in Table 5.

The β₂m may be naturally occurring or modified. A naturally occurring β₂m is a β₂m protein having the amino acid sequence of beta-2 microglobulin isolated from the particular species in question, or a fragment thereof. A polynucleotide sequence encoding a naturally occurring β₂m may be the polynucleotide sequence naturally occurring in the organism or may be any degenerate polynucleotide sequence which encodes the same amino acid sequence. The β₂m may be a variant β₂m, such as an allelic variant of β₂m. Modified β₂m refers to a β₂m having an amino acid sequence that has been modified from a wild-type or naturally occurring beta-2 microglobulin amino acid sequence, or a fragment thereof.

A wild-type or naturally occurring polynucleotide sequence encoding beta-2 microglobulin may be modified by nucleotide substitutions, for example from 1, 2 or 3 to 10, 25 or 50 substitutions. The β₂m sequence may alternatively or additionally be modified by one or more insertions and/or deletions and/or by an extension at either or both ends. A modified β₂m sequence will generally have at least 70%, at least 80%, at least 90%, at least 95%, at least 98% or at least 99% sequence identity to the coding sequence of a wild-type or naturally occurring β₂m sequence (such as that shown in Table 5) over a region of at least 20, preferably at least 30, for instance at least 40, at least 60, more preferably at least 100 contiguous nucleotides or most preferably over the full length of the wild-type or naturally occurring β₂m sequence. Methods of measuring nucleic acid and protein homology are well known in the art. For example the UWGCG Package provides the BESTFIT program which can be used to calculate homology (Devereux et al 1984). Similarly the PILEUP and BLAST algorithms can be used to line up sequences (for example are described in Altschul 1993, and Altschul et al 1990). Many different settings are possible for such programs. In accordance with the invention, the default settings may be used.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides may be used in the invention, or a polynucleotide which has at least 95% sequence identity over 40 nucleotides.

A suitable variant or fragment of a wild-type or naturally occurring β₂m sequence maintains the characteristics of wild-type β₂m. Preferably, the variant or fragment should maintain the ability to assemble with an MHC I heavy chain to form a class I MHC. Preferably, the class I MHC comprising the variant or fragment β₂m should be expressed on the surface of a cell. The assembled MHC comprising the variant or fragment β₂m should retain the ability to present the epitope of the fusion protein at the cell surface. All references to a β₂m sequence below refer to a β₂m sequence or a variant thereof as defined above.

The polynucleotide which is capable of expressing the fusion protein comprises a sequence which encodes the fusion protein, or a precursor thereof, and control sequences which cause expression of the coding sequence. The coding sequence may comprise one or more introns. Where the encoded protein is a precursor of the mature form of fusion protein, the precursor requires post-translational modification to produce the mature form, such as cleavage (proteolysis) of the precursor. In one embodiment the precursor comprises a signal peptide which directs it to the endoplasmic reticulum.

The control sequences are operably linked to the coding sequence of the polynucleotide enabling them to cause expression of the coding sequence in the cells of the host. The control sequences are typically the same as, or substantially similar to, any of the control sequences in the gene of the host or of a virus capable of infecting the host. The control sequences typically comprise a promoter (generally 5′ to the coding sequence) and/or a terminator and/or translation initiation sequence (e.g. GCCACCATGG or GCCCCCATGG) and/or a translational stop codon (e.g. TAA, TAG or TGA) and/or a polyadenylation signal and/or one or more enhancer sequences.

The control sequences may enhance the transcription or translation of the polynucleotide. The control sequences may cause constitutive expression. In a preferred embodiment the control sequences cause tumour-specific or tissue-specific expression (where the tissue is the same tissue as the tumour). In one embodiment this is achieved by use of control sequences whose activity is affected (typically increased) by an agent which is administered to the host. Such an agent may for example be targeted or may localise preferentially to tumour cells or to a particular tissue, and may thus cause the control sequences to only cause expression in tumour cells or in particular tissues. The polynucleotide may be transiently or stably expressed in the target cell.

The polynucleotide may comprise other sequence(s) which aid the transport or activity of the polynucleotide, or which increase the stability of the polynucleotide in the cell. Such a sequence may aid delivery of the polynucleotide to the target cell, for example by causing the polynucleotide to adopt a more compact form or by aiding its association with a targeting or carrier agent. The sequence may aid the integration of the polynucleotide into the genome of the cell or may increase the stability of the polynucleotide in episomal form. In one embodiment the sequence causes replication of the polynucleotide in the cell.

In one embodiment the polynucleotide is also capable of expressing a sequence which encodes a protein that enhances the ability of the fusion protein to restore antigen presentation or which stimulates a CTL response against the epitope of the fusion protein (such as any such protein mentioned herein).

The delivery of the polynucleotide may be targeted, typically to the tumour cells, for example by targeting to tissues of the same type as the tumour cell. The targeting may be achieved by administering the polynucleotide in, or in the vicinity of, the tumour or at a location which ensures in vivo transport of the polynucleotide to the tumour. The polynucleotides may be associated with a moiety that targets delivery of the polynucleotide to a tumour cell. If the polynucleotide is in the form of a vector or associated with a carrier, then such a vector or carrier may act as a targeting moiety.

The moiety which causes the targeting of the polynucleotide is generally able to bind a molecule expressed on surface of the tumour (and which is preferably expressed on the surface of only a small number of or no other cells). The moiety may be natural the ligand/receptor (or fragment and/or homologue thereof of a ligand/receptor expressed on the tumour cells. Suitable ligands/receptors include EGF receptor (e.g. EGFRvIII). The moiety may be an antibody, such as a single-chain antibody.

In one embodiment the ligand/receptor which is targeted is expressed in a different form and/or in increased amounts on the tumour cell, for example in a mutated form. Such proteins include:

GA733-2/epithelial cell adhesion molecule,

receptor type protein tyrosine phosphatase PTPZeta,

EpCAM,

FRFRs (fibroblast growth factor receptors),

EGF receptors,

Her2neu,

RET (receptor tyrosine kinase), and

CEA (carcinoembryonic antigen).

In one embodiment, the polynucleotides of the invention may be administered directly as a naked nucleic acid construct to achieve expression of the fusion protein of the invention. In a further embodiment the polynucleotide may in the form of a viral vector, typically based on a virus which is able to infect the host (preferably also cells of the same tissue as the tumour). Thus the vector may be, or may be derived from, an alphavirus, adenovirus, adeno-associated virus, vaccinia virus, herpes simplex virus, retrovirus (e.g. lentivirus) vector, such as amphotropic or xenotrophic retroviruses derived from Moloney leukemia virus, spleen necrosis virus. The virus vector is typically attenuated, for example replication defective.

The carrier which the polynucleotide may be associated with is typically one which aids the passage of the polynucleotide across the cell membrane. For example, uptake of naked nucleic acid constructs by mammalian cells is enhanced by several known transfection techniques, including those using transfection agents. The carrier may comprise a transfection agent, a cationic agent, for example a cationic lipid, calcium phosphate, DEAE dextran, polyethylenimine (PEI), dendrimers, and lipofectants, for example lipofectam and transfectam, polylysine, a lipid or a precipitating agent (e.g. a calcium salt). The polynucleotide and carrier may be in the form of liposomes or particles. The particle typically has a diameter of 10 to 10⁻³ μm, for example 1 to 10⁻² μm. The carrier may cause the polynucleotide to adopt a more compact form, e.g. a histone. The polynucleotide may be in association with spermidine.

The carrier may be one which can be used to deliver the polynucleotide into the cell, or even into the nucleus, using ballistics techniques. Such a carrier is typically a metal particle, such as a gold particle.

The polynucleotide is generally DNA or RNA, and is typically single or double stranded. The polynucleotide may be chemically modified, typically to enhance resistance to nucleases or to enhance its ability to enter cells. For example, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (MBOs) may be used. NIBOs contain segments of phosphothioate oligodeoxynucleotides and appropriately placed segments of modified oligodeoxy- or oligoribonucleotides. BOs have segments of phosphorothioate linkages and other segments of other modified oligonucleotides, such as methylphosphonate, which is non-ionic, and very resistant to nucleases or 2′-O-alkyloligoribonucleotides.

The polynucleotide may be in substantially isolated form, or it may be in substantially purified form, in which case it will generally comprise at least 80%, 90%, 95%, 98% or 99% of the polynucleotide or dry mass in the preparation. Thus the polynucleotide may be in the form of ‘naked DNA’.

The host may have a CTL response against the peptide epitope of the fusion protein at the time when the fusion protein is administered (due to a previous stimulation) and/or such a response may be stimulated simultaneously, before or after administration of the polynucleotide. The stimulation may be one which is due to a natural infection (bacterial or viral, e.g. influenza virus infection). The stimulation may be due to the administration of an agent that comprises the epitope and optionally also an adjuvant that enhances a CTL response against the epitope.

The present invention further provides a method for selecting a suitable epitope-β₂m fusion protein for use in treating a tumour. The method comprises determining whether the patient has an existing CTL response to an epitope. If the patient has previously been exposed to an epitope, for example due to a natural infection, then a CTL response should be seen in response to exposure to the epitope. A positive CTL response against an epitope indicates that a fusion protein comprising that epitope may be suitable for the treatment of a tumour in that patient, without the need for vaccination with the epitope itself. The CTL response of the patient may still be enhanced by vaccination and/or inclusion of an adjuvant.

The polynucleotide encoding the fusion protein may be administered with or without a vaccination agent. The polynucleotide may be administered with or without first screening for a suitable epitope which the patient is known to produce a CTL response against. Expression of the fusion protein in a tumour, and consequent presentation of the epitope by MHC at the surface of the tumour cells may stimulate a CTL response against those cells. Optionally, a vaccination agent and/or an adjuvant may also be administered. Where the epitope is, for example, a cancer-specific epitope, expression of the fusion protein may stimulate a CTL response against the epitope which may also act against tumour cells of the patient which express that epitope.

Accordingly the invention also provides a product containing a polynucleotide capable of expressing the epitope-β₂m fusion protein and a vaccination agent that stimulates a CTL response against the epitope of the fusion protein for simultaneous, separate or sequential use in the treatment of cancer, for example in the generation of CTL responses against a tumour. In addition the invention provides a composition comprising a polynucleotide capable of expressing the epitope-β₂m fusion protein and a vaccination agent that stimulates a CTL response against the epitope of the fusion protein.

The CTL adjuvant may be capable of causing or augmenting a MHC class II restricted T cell (typically CD4) response which is favourable to the production of a CTL response, such as a Th1 response. Thus the adjuvant may comprise a MHC class II restricted T cell epitope (or a precursor which can be processed in vivo to provide such an epitope). The adjuvant may be a cytokine, such as a cytokine which stimulates a MHC class I restricted T cell response or favourable MHC class II restricted T cell response (e.g. IL-2, IL-7, IL-12 or IFN-γ). The adjuvant may be, for example, CFA (Golding and Scott (1995) Ann. N.Y. Acad. Sci. 754, 126-37), a muramyl dipeptide (e.g. of a mycobacterial cell wall), monophosphoryl lipid A, lipopolysaccharide (e.g. from B. abortus), liposomes, SAF-1 (Golding and Scott, supra), a saponin (e.g. Quil A), keyhole limpet hemocyanin, yeast TY particle, beta 2-microglobulin, mannan (e.g. oxidised mannan), PROVAX (EDEC Ph. Corporation), immunostimulatory DNA sequences (ISS), high molecular weight nonionic block polymers or E. coli heat labile toxin (LT).

The particular route of administration used may aid the stimulating of a CTL response, and thus the polynucleotide or composition may be provided in a form suitable for administering by such a route. The polynucleotide or composition is typically administered by any standard technique. It may be delivered directly, for example, by injection, such as intradermally, subcutaneously or intramuscularly. Intraperitoneal or intravenous routes are preferred. The polynucleotide or compositions may be delivered topically, orally or intranasally or by aerosol or, for example, using a particle bombardment or patch transdermal delivery device. In one embodiment administration is by ballistic means.

Generally a low dose of antigen favours the development of a CTL response. Thus in the method a suitable low dose can be given. The polynucleotide or composition may be provided in an amount and concentration that is suitable for administering to provide an appropriate low dose.

In one embodiment, the vaccination agent may be administered before the polynucleotide of the invention such that the patient develops a CTL response against the epitope of the fusion protein and is capable of producing a CTL response against cells expressing the fusion protein. The time between administration of the vaccination agent and the polynucleotide of the invention should therefore be sufficient to allow the individual to develop an immune response against the epitope of the fission protein. For example; the vaccination agent may be administered one, two, four, six, eight, twelve or more weeks prior to administration of the polynucleotide encoding the fusion protein.

The vaccine may be given in a single dose schedule or in a multiple dose schedule. A multiple dose schedule is one in which a primary course of vaccination may be with 1-10 separate doses, followed by other doses given at subsequent time intervals required to maintain or reinforce the immune response, for example at 1-4 months for a second dose, and if needed a subsequent dose after several months. The dosage regime will also at least in part be determined by the need of the individual and be dependent upon the judgement of the practitioner.

An effective non-toxic amount of the polynucleotide (including in the form of the composition of the invention) and optionally also a vaccination agent that stimulates a CTL response against the peptide epitope of the fusion protein, may be given to a human or non-human patient in need thereof, such as a patient with a cancer or suspected of having cancer. The condition of a patient suffering from a cancer can therefore be improved by such an administration.

Thus the polynucleotide (and optionally also the vaccination agent) is provided for use in a method of treating the human or animal body by therapy. The invention also provides use of the polynucleotide in the manufacture of a medicament for treating cancer for example in the generation of CTL responses against a tumour. The polynucleotide (and generally also the vaccination agent) may be used in a method of treating a host comprising administering the polynucleotide (and generally also the vaccination agent) to the host.

The polynucleotide may be administered in a single dose schedule or in a multiple dose schedule. In one embodiment the subject is given 1, 2, 3 or more separate administrations, each of which is separated by at least 12 hours, 1 day, 2, days, 7 days, 14 days, 1 month or more.

The dose of polynucleotide and/or vaccination may be determined according to various parameters, especially according to the form of the polynucleotide used; the age, weight and condition of the patient to be treated; the capacity of the patient's immune system to produce an immune response; the route of administration; and the required regimen. A physician will be able to determine the required route of administration and dosage for any particular patient. A suitable dose may however be from 1 μg to 10 g, for example from 100 μg to 1 g of the polynucleotide. These values may represent the total amount administered in the complete treatment regimen or may represent each separate administration in the regimen. The quantity of nucleic acid administered per dose is preferably 10 μg or less, such as 1 μg to 10 μg for particle mediated delivery, e.g. ballistics methods, and preferably 1 μg to 10 mg, more preferably 100 μg to 1 mg for other routes, e.g. direct injection.

When the polynucleotide and/or vaccination agent is in the form of a viral vector the amount of virus administered is in the range of from 10⁴ to 10¹² pfu, preferably from 10⁷ to 10¹⁰ pfu. When injected, typically 1-2 ml of virus in a pharmaceutically acceptable suitable carrier or diluent is administered.

The polynucleotide may be in the form of a pharmaceutical composition which comprises the polynucleotide and a pharmaceutically acceptable carrier or diluent. Suitable carriers and diluents include isotonic saline solutions, for example phosphate-buffered saline. Typically the composition is formulated for parenteral, intravenous, intramuscular, subcutaneous, transdermal, intradermal, oral, intranasal, intravaginal, or intrarectal administration.

The following Examples illustrate the invention:

### EXAMPLE 1

Materials and Methods

Cell Lines, Antibodies, Bacterial Strains and Plasmids.

T2 is a lymphoblastoid cell line lacking class I expression due to a defect in the TAP transporter system (1). JY is human B lymphoblastoid cell line (2). DLD-1 is a colorectal adenocarcinoma cell line that is defective in β₂m expression (3). Anti hβ₂m antibody BBM.1 (4) and the anti class I antibody W6/32 (5) were used. The xenotropic cell line PG13 (American Type Culture Collection) was used for the production of recombinant viruses using the retroviral construct pLXSN (Clontech). The bacterial strain used for recombinant proteins expression was BL21(DE3)physS (Novagen). Purified human β₂m was obtained from Sigma.

Construction and Expression of the β₂m-Fusion Molecule

The HLA-A2 restricted influenza matrix epitope (MA 58-66) was linked to human β₂m by a 15 amino acid spacer. An appropriate linker length for the fusion protein was determined by inspection of the HLA-A2/MA peptide crystal structure (PDB code 1HHI) (6). Glycine residues were used for the N terminal portion of the linker to facilitate the continuation of the peptide with minimal disruption to the binding groove as observed in a HLA-A2 crystal structure (PDB code 2CLR) (7). A methionine residue was added to the N-terminus of the protein. The PCR was performed using 5′-primer OX297: 5-GGG GGG CAT ATG GGT ATT TTA GGA TTT GTT TTT ACA TTA GGA GGT GGG GGA GGC GGA TCA GGA GGC TCA GGT GGG TCA GGA GGC ATC CAG CGT ACT CCA AAG ATT CAG G-3′ and 3′-primer OX298: 5′-G ATA GTT AAG TGG GAT CGA GAC ATG TAA GCT TCC CCC-3′. The amplified fragment NdeI-Hind III) was cloned into the expression vector pGMT7, sequenced and used for expression.

The retroviral vector MA-β₂m-pLXSN was generated in a three step cloning strategy. The primers 5′ AAT TCG CCA CCA TGT CTC GCT CCG TGG CCT TAG CTG TGC TCG CGC TAC TCT CTC TTT CTG GCC TCG AGA CTA CTG-3′ and 3′-GAT CCA GTA GTC TCG AGG CCA GAA AGA GAG AGT AGC GCG AGC ACA GCT AAG GCC ACG GAG CGA GAC ATG GTG GCG-5′ were annealed to originate the signal sequence and cloned as a EcoRI-BamHI fragment into the pLXSN construct. A Xho I site was present in the oligo at the 3′ end of the signal sequence.

A plasmid expressing β₂m was used as a template for a PCR reaction using the primer 5′-ATT ATT CTC GAG ATC ATC ATG GAT CCG GCG GAG GCG-3′ encoding the 3′ end of the linker and including the restriction sites for Xho I and BamHI and the primer OX298 described above including a Bgl II site. This fragment was cloned into the Xho-Bgl II sites of the retroviral vector pLXSN and contained at its 5′ end the two sites Xho I and BamHI where the oligos encoding the epitope and part of the linker were cloned into. The two primers constituting this last fragment were 5′-TCG AGG GCG GCA TCC TGG GCT TCG TGT TCA CCC TGG GCG GCG-3′ and 5′-ATT TCG CCA CCA TGT CTC GCT CCT TGG CCT TAG CTG TGC TCG CGC TAC TCT CTC TTT CTG GCC TCG AGA CTA CTG-3′.

Expression, Purification and Tetramerisation of Epitope-β₂m Fusion Protein

The construct containing the HLA-A2 heavy chain and the biotinylation tag was constructed as in (8) The β₂m fusion plasmid (pGMT7) was transformed into the bacterial strain BL21(DE3)phLysS (better done in E. coli FMS 174) and expression induced at OD₆₀₀=0.5 with 0.5 mM IPTG. Bacteria were harvested after 5 h and 10 μl lysate analysed on a 4-20% PAGE. Soluble HLA-peptide tetramers and fusamers were generated as in (8). Refolding of the binary (epitope-β₂m fusion protein/HLA) and ternary (epitope/β₂m/HLA) complex was done in the presence of soluble MHC class I heavy chain and MIA epitope (GILGFVFTL). Recombinant β₂m was used for the ternary complex while the epitope-β₂m fusion protein was used for the binary complex. The refold was concentrated, biotinylated and purified by gel filtration (Superdex 75 column) followed by ion-exchange chromatography. The biotinylation of the complex was tested by ELISA using Extravidin peroxidase conjugate (Sigma) and a TMB substrate for detection (Sigma). The generation of tetrameric class I molecule complexes was achieved by slow addition of extravidin-PE conjugated (Sigma, UK) at a molar ratio of 0.3:1 (extravidin:biotinylated protein) (8).

Retroviral Production and Cell Transduction

The retroviral constructs MA-β₂m-pLXSN and pLXSN were introduced, via calcium phosphate co-precipitation (9) into the xenotropic packaging cell line PG13. After two weeks of selection in G418 [800 μg/ml], supernatant from producing cells was harvested and used for transduction of target cells. The T2 cell line was subsequently transduced with both the vectors while DLD-1 and JY cells were transduced with the retroviral vector MA-β₂m-pLXSN. 5×10⁶ cells were transduced using 5 ml of viral supernatant in presence of protamine 8 μg/ml for 16 hours and subsequently grown in selective medium containing G418 (400, 1600 and 500 μg/ml respectively) for 2 weeks.

Flow Cytometry

For the detection of β₂ in retrovirally transduced cells, 10⁶ cells were washed in PBS/0.1% BSA/1% human serum and incubated with 1 μg of anti-β₂m (BBM.1) antibody for 30 min on ice. The cells were washed twice in PBS/0.1% BSA and incubated with a phycoerythrin conjugated secondary antibody for 30 min on ice. Cells were washed twice and analysed by flow cytometry. For the detection of HLA class I, 1 μg of anti HLA class I antibody (W6/32) was used and the cells were stained as described.

For the tetramer staining, HLA-A2-restricted CTL clones specific for flu matrix protein (clone C3 (10)) or the melanoma antigen melan-A (clone 3G3 (11)) were stained with equivalent doses (0.5 μg) of the fusamer or the tetramer for 15 min at 37° C. After the staining, the cells were washed twice and resuspended in FACS buffer and analysed by flow cytometry.

Western Blotting

Western blotting analysis was performed on the non transduced and retrovirally transduced cell lysate to detect the epitope-β₂m fusion protein. 5×10⁶ cells were pelleted, washed once in PBS and lysed in 60 ml of lysis buffer (20 mM Tris pH 7.6, 10 mM EDTA 100 mM NaCl 0.5% Nonidet P-40) for 20 min on ice. The nuclei were spun down, and 12 μl of supernatants was analysed by SDS page gel electrophoresis and immunoblotted with the monoclonal antibody anti-β₂m BBM1. The intensity of the bands were quantified using Fluorchem™ System (Alpha Innotech Corporation)

⁵¹Chromium Release Assay

After two weeks of selection the transduced cell lines were assayed as target cells in a chromium release assay. The target cells were labeled for 1 hour at 37° C. with 100 mCi per 10⁶ cells Na⁵¹Cr and washed repeatedly. The cells were distributed in round bottom 96-well microtiter plates (5×10³ cells per well) and pulsed with 5 mM of the synthetic peptide for one hour before the addition of a HLA-A2 restricted flu-matrix CTL clone. The effector cells were added at different effector/target ratios and the plates were incubated for 4 hours at 37° C. Supernatants were harvested and ⁵¹Cr-release was measured in a gamma counter. The specific lysis was calculated according to the formula 100×([experimental cpm-background cpm]/[maximum cpm−background cpm]). Background cpm values were determined by incubating target cells alone and maximum values were determined lysing the target cells in presence of Triton 5% x-100. Non transduced cells were used as controls in the same conditions. All the experiments were performed in triplicate.

Biotin-Labeling of Surface Proteins and Immunoprecipitation of β₂m

For biotinylation of surface proteins, 5×10⁷ cells were incubated at 4° C. for 1 hour in 1 ml of PBS-5 mM biotin. The cells were washed once in PBS-glycine 5 mM, and twice in PBS and resuspended in 500 μl of lysis buffer (20 mM Tris pH 7.6, 10 mM EDTA 100 mM NaCl 0.5% Nonidet P-40). β₂m was immunoprecipitated using anti β₂m BBM1 antibody (15 μg/ml) and the immunoprecipitated protein was analysed by western blotting with peroxidase-conjugated streptavidine (1:1000, Sigma).

### EXAMPLE 2

Results

Construction and Expression of Epitope-β₂m Fusion Molecule

We linked the influenza matrix epitope GILGFVFTL to the N-terminus of β₂m via a synthetic linker. The linker was designed by molecular modelling and comprised a Gly-Gly-Ser motif, with the first five amino acids glycines to avoid interference of bulky side chains with the alpha helixes of the binding groove. A linker of 15 amino acids could span the gap between the C-terminus of the epitope and the N-terminus of β₂m. The fusion protein had a molecular mass increase of ca 2.6 kD consistant with the addition of 24 amino acids to β₂m.

Expression and Purification of β₂m Fusion Molecule and Complex Formation with HLA Class I Heavy Chain

The fusion molecule was folded together with soluble HLA-A2 heavy chain with a biotinylation tag on the C-terminus. To test whether the complex contained the fusion molecule and to rule out cleavage of the epitope during handling, we ran fractions of the complex peak on SDS-PAGE. The proteins detected corresponded to the intact fusion molecule and the HLA-A2 heavy chain. We superimposed the Superdex 75 column elution profiles of the classic epitope/HLA complex (ternary complex) and the epitope-β₂m fusion/HLA complex (binary complex). Both complexes showed the same running behaviour, indicating similar size and structure.

Tetramerization of HLA Class I Complexes and Staining of HLA-A2 Restricted Influenza Matrix Clone with Tetramer and Fusamers

The binary complex was biotinylated and multimerized with Streptavidin-PE as for the classic HLA/epitope tetrameric complexes (ternary complexes). The multivalent complexes of these binary and ternary MHC molecules are referred to as fusamers and tetramers. A CTL clone specific for the flu matrix epitope (58-66) stained with the fusamer (Table 2). An irrelevant melanoma CTL clone, also restricted by HLA-A2, did not stain with this reagent. Stability of the fusamer was compared with stability of the tetramer by incubating both reagents at 37° C. for 15 minutes, before staining CTL. The fusamer stained the flu clone as brightly as the tetramer. No decay in the brightness of staining was observed for up to 8 hours for either reagent, hence under these experimental conditions, both reagents seemed to have similar stability at 37° C.

Design of the MA-β₂m-pLXSN Retroviral Vector and Analysis of Transduced Cells

The retroviral vector MA-β₂m-pLXSN was constructed to express the epitope-β₂m fusion protein where the immunodominant HLA-A2 restricted influenza peptide (58-66) is linked via a 15-amino acid linker to the amino-terminus of the human β₂m. The β₂m signal sequence was placed in front of the epitope to target the whole molecule to the endoplasmic reticulum. The virus was produced by transfection of the packaging cell line PA317 and the supernatant was harvested and used to transduce target cells.

TAP-deficient HLA-A2 T2 cells were transduced with the retroviral vector MA-β₂m-pLXSN, stained with W6/32, the conformation-dependent anti-MHC class I antibody, and analysed by flow cytometry. An increase in MHC class I complex on the cell surface was observed (Table 3A), indicating that MA-β₂m had complexed with the MHC class I heavy chain in a conformationally correct manner, independent of the TAP transporter.

A β₂m deficient colorectal adenocarcinoma cell line, DLD-1, was transduced with the retroviral vector, and analysed by flow cytometry for expression of cell surface β₂m and MHC class I. As expected, an increase in cell surface expression of β₂m was observed (Table 3B). Conformationally correct MHC class I complexes were also expressed (Table 3B), confirming the ability of the fusion protein to bind the heavy chain and stabilise the complex.

Characterization of the Expressed Protein

Western blotting analysis was performed to test the expression of the fusion protein and to compare the amount of endogenous and retroviral proteins expressed in transduced cells. Cellular lysates from 1×10⁶ T2 cells and transduced T2 cells were analysed using the monoclonal antibody BBM1 to detect the presence of the epitope-β₂m fusion protein. The two forms of β₂m were detected in the transduced T2 cells as the 12 kD form and the 14 kD fusion protein product of the fusion between hβ₂m, the 15 aa linker and the 9 aa epitope. The analysis showed that the fusion protein was correctly expressed and processed, since the protein had the expected molecular weight. The amount of protein expressed by the retrovirus detected in this assay was about 20 times less than the amount of endogenous β₂m as calculated from the intensity of the bands.

To test the integrity of the fusion protein on the cell surface, transduced DLD-1 cells were surface-labeled with biotin and the epitope-β₂m fusion protein was immunoprecipitated. The results showed that the immunoprecipitated β₂m from the transduced DLD-1 cell line was bigger than the endogenous β₂m immunoprecipitated from Jurkat cells used as control. The retrovirally transduced protein corresponded to a 14 kD protein. This result demonstrated that the protein was correctly transported to the cell surface and not degraded by membrane proteases. A quantitative analysis based on the intensity of the bands showed that the amount of recombinant β₂m expressed was the same as in the control cell line.

The presence of the intact protein on the cell surface was also demonstrated for the fusion protein NP-β₂m (12) and it may be relevant in case of in vivo delivery, where the epitope should not be presented by cells other than the targeted ones. Moreover the fusion protein itself can not be shuffled from one cell to another. When we co-cultured epitope-β₂m fusion expressing cells and non-transduced cells, no fusion protein was detected on the surface of the non-transduced cells analysed by flow cytometry.

Lysis of Transduced Cells by CTL

A ⁵¹chromium release assay was performed using the transduced T2 cells expressing the epitope-β₂m fusion protein. The assay was performed as described above, using a flu-matrix specific CTL clone. The specific killing in absence of epitope was very high for the cells expressing the fusion protein (72%) comparable to the level of lysis obtained for the non transduced cells pulsed with the epitope (70-80%) (Table 4A). The addition of epitope to the transduced cells did not alter significantly the percentage of specific lysis (70-80%).

The values obtained for the T2 cells transduced with a mock virus were comparable to the values obtained for the non transduced cells, demonstrating that the killing is specific for the epitope-β₂m fusion protein.

The retrovirally transduced DLD-1 cells were used as targets in a chromium release assay to assess the ability of the fusion protein to form a functional class I complex in a β₂m negative cell line. The results (Table 4B) showed a high specific lysis of the cells (78%) expressing the epitope-β₂m fusion protein both in the absence and in presence of the epitope. The non transduced cells showed only background lysis.

A lymphoblastoid cell line JY, was transduced with the retroviral vector MA-β₂m-pLXSN and used in the assay as target cells (Table 4C). The specific killing activity using the transduced cells was as high as the normal control (non transduced JY cells) where the epitope was added. On the contrary, the specific lysis in the absence of the epitope was around background levels for the non transduced cell line, while the transduced line showed values comparable with those obtained with the positive controls. In this assay the fusion protein was able to form CTL recognition structures even in the presence of an endogenous HLA class I complex.

The results showed that CTL target structures could be generated with this kind of fusion protein in cells lacking β₂m expression and also in cells expressing β₂m. Moreover, the observation that these targets could be created in a cell line lacking the TAP transporter demonstrated that no epitope processing was required, consistent with the epitope being covalently linked to β₂m as demonstrated in the DLD-1 cell line.

### EXAMPLE 3

Activation of a CTL Response to Peptide-β₂m Fusions In-Vivo.

The ability of peptide->m fusions to elicit a T cell immune response in vivo was tested by in 6 non-humanised mice, by testing the ability of cells transfected with influenza-derived nucleoprotein (NP)-β₂m fusion to prime NP-specific CTL responses in vivo. In order to detect NP-specific CTL it was determined whether or not primed mice were able to reject challenge with recombinant vaccinia viruses expressing NP. The T cell responses were measured using NP specific T cell assays and monitoring the vaccinia titre in the mice ovaries.

Materials and Reagents:

Mice: In both experiments mice of strain C57BL/6 (wildtype laboratory mouse strain) were used.

Virus: Recombinant vaccinia viruses (VV-NP) expressing the MHC class I-restricted (Db) peptide epitope derived from influenza NP (NP366-374), sequence in one letter code ASNENMET, had been generated previously (Beauverger et al, Virology 219: 133-1,9 (1996)).

Target cells: MC57 antigen presenting cells (Leist et al., 1987) express mouse MHC molecules Kb and Db. These were transduced with the NTP-₂m-pLXSN retroviral vector (Uger et al. J. Immunol. 160 1598-1605 (1998)). Expression of the NT-β₂m fusions by the transfected MC57 cells was confirmed by western blot and FACS analysis.

Experimental Details:

Mice 1-4 (M1-M4) were injected, subcutaneously, at Day 0 with 4×10⁶ irradiated MC57 cells transfected the NP-β₂m fusion. These mice were then injected intraperitoneally, at Day 8 with 2×10⁶ pfu (plaque forming units) Vaccinia virus strain VV-NP expressing the NP protein in 200 μl phosphate buffered saline (PBS, standard physiological saline buffer).

Mice 5 & 6 (5-M6) were injected, subcutaneously, at Day 0 with 4×10⁶ irradiated, untransfected MC57 cells. These mice were then injected intraperitoneally, at Day 8 with 2×10⁶ pfu (plaque forming units) Vaccinia virus strain expressing the NP protein plus 200 μl phosphate buffered saline (PBS, standard physiological saline buffer).

To assay CTL activity a modification of a previously described procedure was used (Leist et al. J Immunol 138, 2278-81. (1987)):

On Day 13 the spleens and ovaries were removed from the mice. Lymphocyte cells were washed out of the Spleens. Spleen cells were cultured with conA supernatant. Synthetic peptide corresponding to the NP (366-374) epitope was added to the wells and lymphocytes allowed to proliferate for 5 days.

On Day 5, T cells were counted and CTL killing assays were performed as follows. Chromium51-labelled MC57 cells were prepulsed with NP peptide for 1 hour, then mixed with the cultured mouse lymphocytes at the indicated effector to target ratios. Chromium51 counts from wells of MC57 cells lysed by addition of detergent were set at 100% lysis.

The vaccinia titre in the mice ovaries were measured using a standard plaque-based assay

Results

Vaccinia Clearance from Mice Ovaries

The results generated demonstrate that the NP-β₂m fusion transfected M1-4 mice contained approximately 10⁵-10⁶ less vaccinia VV-NP pfu's than the untransfected M5-M6 mice.

NP Specific CTL Activation in M1-M4

The results generated demonstrate that spleen of one of the NP-β₂m fusion transfected M1-4 mice contained approximately 5 times higher levels of NP specific CTLs than untransfected M5-M6 mice; as measured by HLA specific tetramer staining.

The results presented in this example demonstrate the ability of tumour cells transfected with NP-β₂m fusion to elicit an NP specific CTL response.

TABLE 1
EBV epitopes
FLRGRAYGL HLA-B8 (latent epitope)
RRIYDLIEL HLA-B27 (latent cycle Ag EBNA 3C 258-266)
QAKWRLQTL HLA-B8 (latent)
RAKFQLLQ (190-197) (immediate early trans
activator protein BZLF1) HLA-B8
GLCTLVAML (280-288) BMLF1 HLA-A201
CMV epitopes
NLVPMVATV (495-503) lower matrix protein pp65 HLA-A20201
TPRVTGGGAM (417-426) lower matrix protein pp65 HLA-B0702
Influenza virus epitope
GILGFVFTL Influenza matrix epitope (58-66) HLA-A201
Tumor epitopes

| Antigens | HLA restriction | Epitopes |
|---|---|---|
| Tyrosinase | HLA-A2 | MLLAVLYCL |
| | HLA-A2 | YMNGTMSQV |
| | HLA-B44 | SEIWRDIDF |
| | HLA-A24 | AFLPWHRLF |
| | HLA-A1 | KCDICTDEY |
| MART-1/Melan-A | HLA-A1 | SSDYVIPIGTY |
| | HLA-A2 | AAGIGILTV |
| | HLA-A2 | EAAGIGILTV |
| Gp100 | HLA-B45 | AEEAAGIGILTV |
| | HLA-A2 | KTWGQYWQV |
| | HLA-A2 | ITDQVPFSV |
| | HLA-A2 | YLEPGPVTA |
| | HLA-A2 | LLDGTATLRL |
| | HLA-A2 | VLYRYGSFSV |
| | HLA-A2 | RLMKQDFSV |
| | HLA-A2 | RLPRIFCSC |
| | HLA-A3 | LIYRRRLMK |
| | HLA-A3 | ALLAVGATK |
| | HLA-A2 | VYFFLPDHL |
| Gp75/TRP-1 | HLA-A31 | MSLQRQFLR |
| TRP-2 | HLA-A31 | LLPGGRPYR |
| | HLA-A31 | LLPGGRPYR |
| | HLA-A2 | SVYDFFVWL |
| | HLA-A68 | EVISCKLIKR |
| MAGE-1 | HLA-A1 | EADPTGHSY |
| | HLA-Cw16 | SAYGEPRKL |
| MAGE-3 | HLA-A1 | EVDPIGHLY |
| | HLA-A2 | FLWGPRALV |
| | HLA-B44 | MEVDPIGHLY |
| GAGE | HLA-Cw6 | YRPRPRRY |
| BAGE | HLA-Cw16 | AARAVFLAL |
| RAGE | HLA-B7 | SPSSNRIRNT |
| NY-ESO-1/CAG3 | | |
| ORF1 | HLA-A2 | (Q)SLLMWITQC(FL) |
| | HLA-A31 | ASGPGGGAPR |
| ORF2 | HLA-A31 | LAAQERRVPR |
| LAGE/CAMEL | | |
| ORF2 | HLA-A2 | MLMAQEALAFL |

TABLE 2

| | Staining |
|---|---|
| Influenza clone unstained | 0% |
| Melanoma clone unstained | 1.2% |
| Influenza clone + fusamer | 93.5% |
| Influenza clone unstained | 0% |
| Influenza clone + fusamer | 89.6% |
| Influenza clone + tetramer | 68% |

TABLE 3A

| | W632 |
|---|---|
| T2 | 10.84% |
| T2 + MA-β₂m-pLXSN | 39.40% |
| B cells | 59.16% |

TABLE 3B

| | W632 | BBM1 |
|---|---|---|
| DLD-1 | 39.7% | 10.57% |
| DLD-1 + MA-β₂m- | 88.92% | 62.59% |
| pLXSN | | |

TABLE 4A

| | T2 % | T2 + pLXSN | T2 + MA-β₂m-pLXSN |
|---|---|---|---|
| | specific killing | % specific killing | % specific killing |
| 1:1 + peptide | 50 | 70.26 | 50.39 |
| 1:1 − peptide | 3.1 | 14.9 | 72 |
| 3:1 + peptide | 68.76 | 80.33 | 67.3 |
| 3:1 − peptide | 18.7 | 22.16 | 72.79 |

TABLE 4B

| | | DLD-1 + |
|---|---|---|
| | DLD-1 | MA-β₂m-pLXSN % |
| | % specific killing | specific killing |
| 1:1 + peptide | 12.47 | 28.3 |
| 3:1 + peptide | 10.69 | 59.6 |
| 5:1 + peptide | 17.5 | 69.2 |
| 1:1 − peptide | 11.59 | 28.39 |
| 3:1 − peptide | 15 | 64 |
| 5:1 − peptide | 18.54 | 79.08 |

TABLE 4C

| | JY | JY + MA-β₂m-pLXSN |
|---|---|---|
| | % specific killing | % specific killing |
| 1:1 + peptide | 51 | 47.8 |
| 3:1 + peptide | 65.64 | 67 |
| 7:1 + peptide | 75.85 | 73.75 |
| 1:1 − peptide | 0.46 | 47.69 |
| 3:1 − peptide | 6.06 | 58.75 |
| 7:1 − peptide | 8.09 | 71.86 |

TABLE 5
Human β₂m sequences

| | | | | | |
|---|---|---|---|---|---|
| DNA sequence | | | | | |
| atccagcgta | ctccaaagat | tcaggtttac | tcacgtcatc | cagcagagaa | tggaaagtca |
| aatttcctga | attgctatgt | gtctgggttt | catccatccg | acattgaagt | tgacttactg |
| aagaatggag | agagaattga | aaaagtggat | cattcagact | tgtctttcag | caaggactgg |
| tctttctatc | tcttgtacta | cactgaattc | acccccactg | aaaaagatga | gtatgcctgc |
| cgtgttaacc | atgtgacttt | gtcacagccc | aagatagtta | agtgggatcg | agacatgaga |
| Protein sequence | | | | | |
| IQRTPKIQVY | SRHPAENGKS | NFLNCYVSGF | HPSDIEVDLL | KNGERIEKVD | HSDLSFSKDW |
| SFYLLYYTEF | TPTEKDEYAC | RVNHVTLSQP | KIVKWDRDMR | | |

### REFERENCES

1. Salter, R. D., D. N. Howell, and P. Cresswell. 1985. Genes regulating HLA class I antigen expression in T-B lymphoblast hybrids. Immunogenetics 21:235.

2. Spits, H., M. Breuning, P. Ivanyi, C. Russo, and J. E. de Vries. 1982. In vitro-isolated human cytotoxic T-lymphocyte clones detect variations in serologically defined HLA antigens. Immunogenetics 16:503.

3. Bicknell, D. C., A. Rowan, and W. F. Bodmer. 1994. Beta 2-microglobulin gene mutations: a study of established colorectal cell lines and fresh tumors. Proc Natl Acad Sci USA 91:4751.

4. Parham, P., M. J. Androlewicz, N. J. Holmes, and B. E. Rothenberg. 1983. Arginine 45 is a major part of the antigenic determinant of human beta 2-microglobulin recognized by mouse monoclonal antibody BBM.1. J Biol Chem 258:6179.

5. Brodsky, F. M., and P. Parham. 1982. Monomorphic anti-HLA-A,B,C monoclonal antibodies detecting molecular subunits and combinatorial determinants. J Immunol 128:129.

6. Collins, E. J., D. N. Garboczi, and D. C. Wiley. 1994. Three-dimensional structure of a peptide extending from one end of a class I MHC binding site. Nature 371:626.

7. Madden, D. R., D. N. Garboczi, and D. C. Wiley. 1993. The antigenic identity of peptide-MHC complexes: a comparison of the conformations of five viral peptides presented by HLA-A2 [published erratum appears in Cell 1994 Jan. 28;76(2):following 410]. Cell 75:693.

8. Altman, J. D., P. A. H. Moss, P. J. R. Goulder, D. H. Barouch, M. G. McHeyzer-Williams, J. I. Bell, A. J. McMichael, and M. M. Davis. 1996. Phenotypic analysis of antigen-specific T lymphocytes [published erratum appears in Science 1998 Jun. 19;280(5371):1821]. Science 274:94.

9. Sambrook, J., E. Fritsch, and T. Maniatis. 1989. Molecular cloning—laboratory manual. Cold Spring Harbor Laboratory Press.

10. Dunbar, P. R., G. S. Ogg, J. Chen, N. Rust, P. van der Bruggen, and V. Cerundolo. 1998. Direct isolation, phenotyping and cloning of low-frequency antigen-specific cytotoxic T lymphocytes from peripheral blood. Curr Biol 8:413.

11. Dunbar, P. R., J. L. Chen, D. Chao, N. Rust, H. Teisserenc, G. S. Ogg, P. Romero, P. Weynants, and V. Cerundolo. 1999. Cutting edge: rapid cloning of tumor-specific CTL suitable for adoptive immunotherapy of melanoma. J Immunol 162:6959.

12. Uger, R. A., and B. H. Barber. 1998. Creating CTL targets with epitope-linked beta 2-microglobulin constructs. J Immunol 160:1598.

1. A polynucleotide capable of expressing a epitope-β₂m fusion protein; for use in the generation of cytotoxic T lymphocyte (CTL) responses against a tumour.

2. A polynucleotide capable of expressing a epitope-β2m fusion protein; for use in a method of restoring antigen presentation in the tumour of a host.

3. A polynucleotide according to claim 1 or 2 in which the epitope is not a cancer epitope.

3. A polynucleotide according to any one of the preceding claims in which the epitope comprises a viral epitope.

4. A polynucleotide according to claim 4 in which the epitope is an epitope which occurs in Epstein-Barr virus, cytomegalovirus or influenza virus.

6. A polynucleotide according to any one of the preceding claims which is in the form of a viral vector.

7. A polynucleotide according to any one of the preceding claims which is associated with a moiety that targets delivery of the polynucleotide to a tumour cell.

8. A polynucleotide according to any one of the preceding claims in association with a pharmaceutically acceptable diluent or carrier.

9. A polynucleotide according to any one of the preceding claims in which the host has a CTL response against the epitope.

10. In combination, a polynucleotide capable of expressing a epitope-β₂m fusion protein and a vaccination agent that stimulates a CTL response against the epitope of the fusion protein for simultaneous, separate or sequential use in the treatment of cancer.

11. A composition comprising a polynucleotide capable of expressing a epitope-β₂m fusion protein and a vaccination agent that stimulates a CTL response against the epitope of the fusion protein.

12. A polynucleotide or fusion protein as defined in claim 7.

13. A method of treating a tumour comprising:
administering a polynucleotide capable of expressing a epitope-β₂m fusion protein, and optionally also
administering a vaccination agent that stimulates a CTL response against the epitope of the fusion protein.


SEQUENCE LISTING (57 sequences)

SEQ ID NO: 1 - protein, 15 aa, Homo sapiens
GGSGGGGSGGSGGSG

SEQ ID NO: 2 - DNA, 10 bp, Homo sapiens
gccaccatgg

SEQ ID NO: 3 - DNA, 10 bp, Homo sapiens
gcccccatgg

SEQ ID NO: 4 - DNA, 109 bp, Artificial Sequence
  Primer
ggggggcatatgggtattttaggatttgtttttacattaggaggtgggggaggcggatcaggaggctcaggtgggtcaggaggcatccagcgtactccaaagattcagg

SEQ ID NO: 5 - DNA, 37 bp, Artificial Sequence
  Primer
gatagttaagtgggatcgagacatgtaagcttccccc

SEQ ID NO: 6 - DNA, 75 bp, Artificial Sequence
  Primer
aattcgccaccatgtctcgctccgtggccttagctgtgctcgcgctactctctctttctggcctcgagactactg

SEQ ID NO: 7 - DNA, 75 bp, Artificial Sequence
  Primer
gcggtggtacagagcgaggcaccggaatcgacacgagcgcgatgagagagaaagaccggagctctgatgacctag

SEQ ID NO: 8 - DNA, 36 bp, Artificial Sequence
  Primer
attattctcgagatcatcatggatccggcggaggcg

SEQ ID NO: 9 - DNA, 42 bp, Artificial Sequence
  Primer
tcgagggcggcatcctgggcttcgtgttcaccctgggcggcg

SEQ ID NO: 10 - DNA, 75 bp, Artificial Sequence
  Primer
atttcgccaccatgtctcgctccttggccttagctgtgctcgcgctactctctctttctggcctcgagactactg

SEQ ID NO: 11 - protein, 9 aa, H. Influenza
GILGFVFTL

SEQ ID NO: 12 - protein, 9 aa, Epstein Barr Virus
FLRGRAYGL

SEQ ID NO: 13 - protein, 9 aa, Epstein Barr Virus
RRIYDLIEL

SEQ ID NO: 14 - protein, 9 aa, Epstein Barr Virus
QAKWRLQTL

SEQ ID NO: 15 - protein, 8 aa, Epstein Barr Virus
RAKFQLLQ

SEQ ID NO: 16 - protein, 9 aa, Epstein Barr Virus
GLCTLVAML

SEQ ID NO: 17 - protein, 9 aa, Cytomegalovirus
NLVPMVATV

SEQ ID NO: 18 - protein, 10 aa, Cytomegalovirus
TPRVTGGGAM

SEQ ID NO: 19 - protein, 9 aa, Influenza virus
GILGFVFTL

SEQ ID NO: 20 - protein, 9 aa, Homo sapiens
MLLAVLYCL

SEQ ID NO: 21 - protein, 9 aa, Homo sapiens
YMNGTMSQV

SEQ ID NO: 22 - protein, 9 aa, Homo sapiens
SEIWRDIDF

SEQ ID NO: 23 - protein, 9 aa, Homo sapiens
AFLPWHRLF

SEQ ID NO: 24 - protein, 9 aa, Homo sapiens
KCDICTDEY

SEQ ID NO: 25 - protein, 11 aa, Homo sapiens
SSDYVIPIGTY

SEQ ID NO: 26 - protein, 9 aa, Homo sapiens
AAGIGILTV

SEQ ID NO: 27 - protein, 10 aa, Homo sapiens
EAAGIGILTV

SEQ ID NO: 28 - protein, 12 aa, Homo sapiens
AEEAAGIGILTV

SEQ ID NO: 29 - protein, 9 aa, Homo sapiens
KTWGQYWQV

SEQ ID NO: 30 - protein, 9 aa, Homo sapiens
ITDQVPFSV

SEQ ID NO: 31 - protein, 9 aa, Homo sapiens
YLEPGPVTA

SEQ ID NO: 32 - protein, 10 aa, Homo sapiens
LLDGTATLRL

SEQ ID NO: 33 - protein, 10 aa, Homo sapiens
VLYRYGSFSV

SEQ ID NO: 34 - protein, 9 aa, Homo sapiens
RLMKQDFSV

SEQ ID NO: 35 - protein, 9 aa, Homo sapiens
RLPRIFCSC

SEQ ID NO: 36 - protein, 9 aa, Homo sapiens
LIYRRRLMK

SEQ ID NO: 37 - protein, 9 aa, Homo sapiens
ALLAVGATK

SEQ ID NO: 38 - protein, 9 aa, Homo sapiens
VYFFLPDHL

SEQ ID NO: 39 - protein, 9 aa, Homo sapiens
MSLQRQFLR

SEQ ID NO: 40 - protein, 9 aa, Homo sapiens
LLPGGRPYR

SEQ ID NO: 41 - protein, 9 aa, Homo sapiens
SVYDFFVWL

SEQ ID NO: 42 - protein, 10 aa, Homo sapiens
EVISCKLIKR

SEQ ID NO: 43 - protein, 9 aa, Homo sapiens
EADPTGHSY

SEQ ID NO: 44 - protein, 9 aa, Homo sapiens
SAYGEPRKL

SEQ ID NO: 45 - protein, 9 aa, Homo sapiens
EVDPIGHLY

SEQ ID NO: 46 - protein, 9 aa, Homo sapiens
FLWGPRALV

SEQ ID NO: 47 - protein, 10 aa, Homo sapiens
MEVDPIGHLY

SEQ ID NO: 48 - protein, 8 aa, Homo sapiens
YRPRPRRY

SEQ ID NO: 49 - protein, 9 aa, Homo sapiens
AARAVFLAL

SEQ ID NO: 50 - protein, 10 aa, Homo sapiens
SPSSNRIRNT

SEQ ID NO: 51 - protein, 12 aa, Homo sapiens
QSLLMWITQCFL

SEQ ID NO: 52 - protein, 10 aa, Homo sapiens
ASGPGGGAPR

SEQ ID NO: 53 - protein, 10 aa, Homo sapiens
LAAQERRVPR

SEQ ID NO: 54 - protein, 11 aa, Homo sapiens
MLMAQEALAFL

SEQ ID NO: 55 - protein, 9 aa, Influenza virus
ASNENMETM

SEQ ID NO: 56 - DNA, 300 bp, Homo sapiens
  CDS (1)...(300)
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggatcattcagacttgtctttcagcaaggactggtctttctatctcttgtactacactgaattcacccccactgaaaaagatgagtatgcctgccgtgttaaccatgtgactttgtcacagcccaagatagttaagtgggatcgagacatgaga

SEQ ID NO: 57 - protein, 100 aa, Homo sapiens
IQRTPKIQVYSRHPAENGKSNFLNCYVSGFHPSDIEVDLLKNGERIEKVDHSDLSFSKDWSFYLLYYTEFTPTEKDEYACRVNHVTLSQPKIVKWDRDMR